(19) United States
(12) Patent Application Publication
(10) Pub. No.: US 2003/0119877 A1
(43) Pub. Date: Jun. 26, 2003

(54) Pyridine derivatives useful as cyclooxygenase inhibitor

(76) Inventors: Junya Ishida, Osaka (JP); Hirofumi Yamamoto, Osaka (JP); Nobukiyo Konishi, Osaka (JP); Masataka Morita, Osaka (JP); Katsuya Nakamura, Osaka (JP); Susumu Miyata, Osaka (JP); Takehiro Ochi, Osaka (JP); Yoshiaki Morita, Osaka (JP); Eiji Yoshimi, Osaka (JP); Kanae Kuroda, Osaka (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/204,003

(22) PCT Filed: Dec. 21, 2001
(86) PCT No.: PCT/JP01/11241

(30) Foreign Application Priority Data
Jan. 2, 2001 (AU) PR2372
Feb. 9, 2001 (AU) PR3007
Jul. 30, 2001 (AU) PR6697

(51) Int. Cl. A61K 31/444; A61K 31/44; C07D 213/84; C07D 213/46; C07D 213/24

(52) U.S. Cl. 514/332; 514/344; 514/345; 514/354; 514/357; 546/261; 546/286; 546/294; 546/314; 546/329; 546/334

(57) ABSTRACT

A compound of the formula (I), wherein R¹ is hydrogen, halogen, carbymoyl, cyano, formuly, or lower alkyl optionally substituted with halogen, amino or a protected amino, R² is hydrogen, halogen, cyano or lower alkoxy, R³ is phenyl or pyridyl, each of which is substituted with lower alkoxy, and R⁴ is lower alkoxy; provided that either R¹ or R² is hydrogen, then the other is other than hydrogen, or its salts, which are useful as a medicament.

### TECHNICAL FIELD

This invention relates to novel pyridine compounds having pharmacological activity, to a process for their production and to a pharmaceutical composition containing the same.

### BACKGROUND ART

The presence of two cyclooxygenase isoenzymes, cyclooxygenase-I (COX-I) and cyclooxygenase-II (COX-II)is known (Proc. Nat. Acad. Sci. USA 88, 2692-2696 (1991)).

Traditional non steroidal anti-inflammatory compounds (NSAIDs) have inhibiting activities of both COX-I and COX-II (J. Biol. Chem., 268, 6610-6614 (1993), etc). The therapeutic use thereof involves undesired effects on the gastrointestinal tract, such as bleeding, erosions, gastric and intestinal ulcers, etc.

It was reported that selective inhibition of COX-II shows anti-inflammatory and analgesic activities comparable with conventional NSAIDs but with a lower incidence of some gastrointestinal undesired effects (Pro. Nat. Acad. Sci. USA, 91, 3228-3232 (1994)). Accordingly, various selective COX-II inhibitors have been prepared. However, it was reported that those “selective COX-II inhibitor” show some side-effects on kidney and/or insufficient efficacy on acute pains.

Further, some compounds such as SC-560, mofezolac, etc, which have certain selective inhibiting activity against COX-II WO98/57910 shows some compounds having such activity. However, their selectivity of inhibiting COX-I does not seem to be enough to use them as a clinically acceptable and satisfactory analgesic agent due to their gastrointestinal disorders.

And further, some pyridine derivatives having cyclooxygenase-II inhibiting activity have already been known by WO96/24584 and WO98/03484.

### DISCLOSURE OF INVENTION

This invention relates to pyridine compounds, which have pharmaceutical activity such as cyclooxygenase (hereinafter described as COX) inhibiting activity, to a process for their production, to a pharmaceutical composition containing the same and to a use thereof.

Accordingly, one object of this invention is to provide the pyridine compounds, which have a COX inhibiting activity.

Another object of this invention is to provide a process for production of the pyridine compounds.

A further object of this invention is to provide a pharmaceutical composition containing, as active ingredients, the pyridine compounds.

Still further object of this invention is to provide a use of the pyridine compounds for manufacturing a medicament for treating or preventing various diseases.

The new pyridine compounds of this invention can be represented by the following general formula (I):

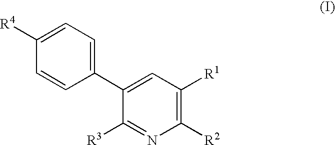

wherein

R¹ is hydrogen, halogen, carbamoyl, cyano, formyl, or lower alkyl optionally substituted with halogen, amino or a protected amino;

R² is hydrogen, halogen, cyano or lower alkoxy;

R³ is phenyl or pyridyl, each of which is substituted with lower alkoxy; and

R⁴ is lower alkoxy;

provided that either R¹ or R²is hydrogen, then the other is other than hydrogen,

or its salts

The compounds (I) or its salts are able to be produced in a similar manner to the general processes and Examples shown below

Process 1

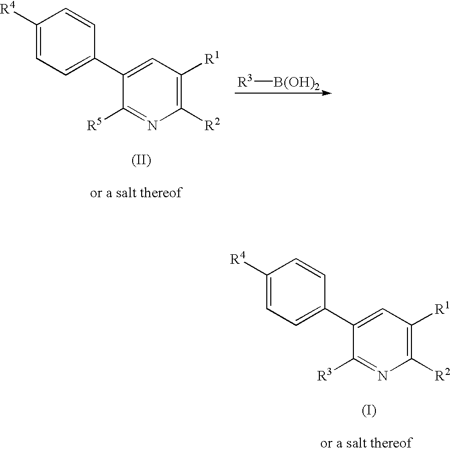

in which R¹, R², R³ , and R⁴ are each as defined above, and R⁵ is a leaving group, such as halogen, trifluoromethanesulfonyloxy, etc.

Process 2

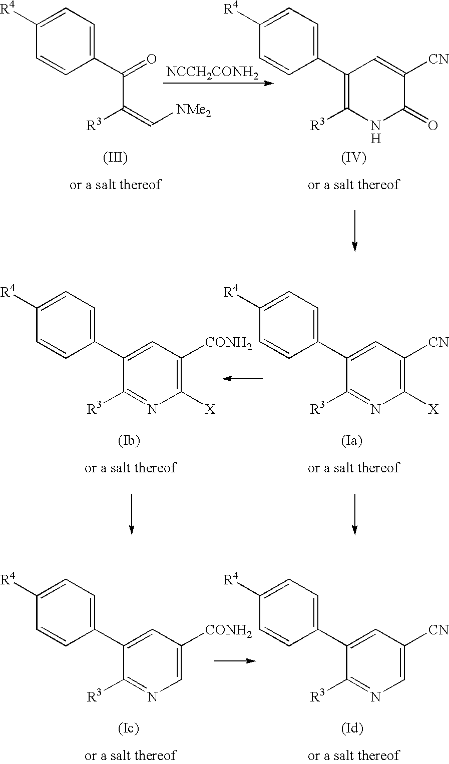

, in which R³ and R⁴ are each as defined above, and

X is halogen.

The compounds of formula (I) may contain one or more asymmetric centers and thus they can exist as enantiomers or diastereoisomers. This invention includes both mixtures and separate individual isomers.

The compounds of the formula (I) may also exist in tautomeric forms and the invention includes both mixtures and separate individual tautomers.

The compounds of the formula (I) and its salts can be in a form of a solvate, which is included within the scope of the present invention. The solvate preferably include a hydrate and an ethanolate.

Also included in the scope of invention are radiolabelled derivatives of compounds of formula (I) which are suitable for biological studies.

In the above and subsequent description of the present specification, suitable examples of the various definitions to be included within the scope of the invention are explained in detail in the following.

The term “lower” is intended to mean a group having 1 to 6 carbon atom(s), unless otherwise provided.

Suitable “lower alkyl” and lower alkyl moiety in the term “lower alkoxy” may be a straight or branched one, such as methyl, ethyl, propyl, isopropyl, butyl, isobutyl, tert-butyl, pentyl, hexyl, or the like, in which preferable one is methyl.

Suitable “halogen” may be fluoro, chloro, bromo or iodo or the like, which preferable one is chloro.

Suitable amino-protective group in the term “a protected amino” is acyl (such as, lower alkanoyl, carbamoyl, etc), lower alkyl, etc.

Suitable “lower alkyl optionally substituted with halogen, amino or a protected amino” is lower alkyl; lower alkyl substituted with halogen; lower alkyl substituted with amino; or lower alkyl substituted with a protected amino.

More preferable “lower alkyl substituted with halogen” is difluoromethyl, trifluoromethyl, or the like, in which the most preferable one is difluoromethyl.

More preferable “lower alkyl substituted with amino” is aminomethyl, aminoethyl, or the like, in which the most preferable one is aminomethyl.

More preferable “lower alkyl substituted with a protected amino” is mono- or di-lower alkylamino(lower)alkyl, such as methylaminomethyl, dimethylaminomethyl, etc; lower alkanoylamino(lower)alkyl, such as acetylaminomethyl; methylcarbamoylaminomethyl.

Suitable “phenyl or pyridyl, each of which is substituted with lower alkoxy” is 4-(lower)alkoxyphenyl or 6-(lower)alkoxy-pyridine-3-yl, in which suitable lower alkyl moiety may be the same as the before-mentioned lower alkyl. The most preferable one is 4-methoxyphenyl or 6-methoxypyridine-3-yl.

Among the compound (I), the following compounds are exemplified as the more preferable ones.

1) The compound, in which

R¹ is halogen, carbamoyl, cyano, formyl, or lower alkyl optionally substituted with halogen, amino or a protected amino;

R² is hydrogen;

R³ is phenyl substituted with lower alkoxy, or pyridyl substituted with lower alkyl; and

R⁴ is lower alkoxy.

2) The compound, in which

R¹ is hydrogen;

R² is halogen, cyano or lower alkoxy;

R³ is phenyl substituted with lower alkoxy; and

R⁴ is lower alkoxy.

3) The compound, in which

R¹ is cyano, or lower alkyl optionally substituted with halogen, amino or a protected amino;

R² is halogen, cyano or lower alkoxy;

R³ is phenyl substituted with lower alkoxy; and

R⁴ is lower alkoxy.

Suitable salts of the compounds (I) are pharmaceutically acceptable conventional nontoxic salts and include a metal salt such as an alkali metal salt (e.g., sodium salt, potassium salt, etc.) and an alkaline earth metal salt (e.g., calcium salt, magnesium salt, etc.), an ammonium salt, an organic base salt (e.g., trimethylamine salt, triethylamine salt, pyridine salt, picoline salt, dicyclohexylamine salt, etc.), an organic acid salt (e.g., acetate, maleate, tartrate, methanesulfonate, benzenesulfonate, formate, toluenesulfonate, trifluoroacetate, etc.), an inorganic acid salt (e.g., hydrochloride, hydrobromide, sulfate, phosphate, etc.), a salt with an amino acid (e.g., arginine, aspartic acid, glutamic acid, etc.), or the like.

In order to illustrate the usefulness of the object compounds (I), the pharmacological test data of the compounds (I) are shown in the following.

[A] Analgesic Activity

Effect on Adjuvant Arthritis in Rats:

(i)Test Method:

Arthritis was induced by injection of 0.5 mg of Mycobacterium tuberculosis (Difco Laboratories, Detroit, Mich.) in 50 μl of liquid paraffin into the right hind footpad of Lewis rats aged 7 weeks. Analgesic activity of a single dose of agents in arthritic rats was studied. Arthritic rats were randomized and grouped (n=10) for drug treatment based on pain threshold of left hind paws and body weight on day 22. Drugs (Test compounds) were administered and the pain threshold was measured 2 hr after drug administration. The intensity of hyperalgesia was assessed by the method of Randall-Selitto. The mechanical pain threshold of the left hind paw (uninjected hind paw) was determined by compressing the ankle joint with a balance pressure apparatus (Ugo Basile Co. Ltd., Varese, Italy). The threshold pressure of rats squeaking or struggling was expressed in grams. The threshold pressure of rats treated with drugs was compared with that of non-treated rats. A dose showing the ratio of 1.5 is considered to be the effective dose.

(ii) Test Results

| Test compound | Dose | The coefficient of |
|---|---|---|
| (Example No.) | (mg/kg) | analgesic |
| Example 4 | 3.2 | >1.5 |
| Example 13-(5) | 3.2 | >1.5 |

[B] Inhibiting activity against COX-I and COX-II
(Whole Blood Assay):
(i) Test Method:
Whole blood assay for COX-I

Fresh blood was collected by syringe without anticoagulants from volunteers with consent. The subjects had no apparent inflammatory conditions and had not taken any medication for at least 7 days prior to blood collection. 500 μl aliquots of human whole blood were immediately incubated with 2 μl of either dimethyl sulfoxide vehicle or a test compound at final concentrations for 1 hr at 37° C. to allow the blood to clot. Appropriate treatments (no incubation) were used as blanks. At the end of the incubation, 5 μl of 250 mM Indomethacin was added to stop the reaction. The blood was centrifuged at 6000×g for 5 min at 4° C. to obtain serum. A 100 μl aliquot of serum was mixed with 400 μl methanol for protein precipitation. The supernatant was obtained by centrifuging at 6000×g for 5 min at 4° C. and was assayed for TXB₂ using an enzyme immunoassay kit according to the manufacturer's procedure. For a test compound, the results were expressed as percent inhibition of thromboxane B₂ (TXB₂) production relative to control incubations containing dimethyl sulfoxide vehicle. The data were analyzed by that a test compound at the indicated concentrations was changed log value and was applied simple linear regression. IC₅₀ value was calculated by least squares method.

Whole blood assay for COX-II

Fresh blood was collected in heparinized tubes by syringe from volunteers with consent. The subjects had no apparent inflammatory conditions and had not taken any medication for at least 7 days prior to blood collection. 500 μl aliquots of human whole blood were incubated with either 2 μl dimethyl sulfoxide vehicle or 2 μl of a test compound at final concentrations for 15 min at 37° C. This was followed by incubation of the blood with 10 μl of 5 mg/ml lipopolysaccharide for 24 hr at 37° C. for induction of COX-II. Appropriate PBS treatments (no LPS) were used as blanks. At the end of the incubation, the blood was centrifuged at 6000×g for 5 min at 4° C. to obtain plasma. A 100 μl aliquot of plasma was mixed with 400 μl methanol for protein precipitation. The supernatant was obtained by centrifuging at 6000×g for 5 min at 4° C. and was assayed for prostagrandin E₂ (PGE₂) using a radioimmunoassay kit after conversion of PGE₂ to its methyl oximate derivative according to the manufacturer's procedure. For a test compound, the results were expressed as percent inhibition of PGE₂ production relative to control incubations containing dimethyl sulfoxide vehicle. The data were analyzed by that a test compound at the indicated concentrations was changed log value and was applied simple linear regression. IC₅₀ value was calculated by least squares method.

(ii) Test Results:

| Test Compound | COX-I | COX-II |
|---|---|---|
| (Example No.) | IC₅₀ (μM) | IC₅₀ (μM) |
| Example 1-(3) | <0.1 | >1 |
| Example 4 | <0.1 | >1 |
| Example 6 | <0.1 | >1 |
| Example 11-(4) | <0.1 | >1 |

It appeared, from the above-mentioned Test Results, that the compound (I) or pharmaceutically acceptable salts thereof of the present invention have an inhibiting activity against COX, particularly a selective inhibiting activity against COX-I.

Additionally, it was further confirmed that the compounds (I) of the present invention lack undesired side-effects of non-selective NSAIDs, such as gastrointestinal disorders, bleeding, renal toxicity, cardiovascular affection, etc.

The object compound (I) or pharmaceutically acceptable salts thereof of this invention possesses COX inhibiting activity and possesses strong anti-inflammatory, antipyretic, analgesic, antithrombotic, anti-cancer activities, and so on. The object compound (I) and pharmaceutically acceptable salt thereof, therefore, are useful for treating and/or preventing COX mediated diseases, inflammatory conditions, various pains, collagen diseases, autoimmune diseases, various immunological diseases, thrombosis, cancer and neurodegenerative diseases in human beings or animals by using administered systemically or topically. More particularly, the object compound (I) and pharmaceutically acceptable salts thereof are useful for treating and/or preventing inflammation and acute or chronic pain in joint and muscle [e.g. rheumatoid arthritis, rheumatoid spondylitis, osteoarthritis, gouty arthritis, juvenile arthritis, etc.], inflammatory skin condition [e.g. sunburn, burns, eczema, dermatitis, etc.], inflammatory eye condition [e.g. conjunctivitis, etc.], lung disorder in which inflammation is involved [e.g. asthma, bronchitis, pigeon fancier's disease, farmer's lung, etc.], condition of the gastrointestinal tract associated with inflammation [e.g. aphthous ulcer, Chrohn's disease, atopic gastritis, gastritis varialoforme, ulcerative colitis, coeliac disease, regional ileitis, irritable bowel syndrome, etc.], gingivitis, inflammation, pain and tumescence after operation or injury, pyrexia, pain and other conditions associated with inflammation, particularly those in which lipoxygenase and cyclooxygenase products are a factor, systemic lupus erythematosus, scleroderma, polymyositis, tendinitis, bursitis, periarteritisnodose, rheumatic fever, Sjogren's syndrome, Behcet disease, thyroiditis, type I diabetes, nephrotic syndrome, aplastic anemia, myasthenia gravis, uveitis contact dermatitis, psoriasis, Kawasaki disease, sarcoidosis, Hodgkin's disease, Alzheimers disease, or the like. Additionally, the object compound (I) or a salt thereof is expected to be useful as therapeutical and/or preventive agents for cardiovascular or cerebrovascular diseases, the diseases caused by hyperglycemia and hyperlipemia.

For therapeutic purpose, the compound (I) and a pharmaceutically acceptable salt thereof of the present invention can be used in a form of pharmaceutical preparation containing one of said compounds as an active ingredient, in admixture with a pharmaceutically acceptable carrier such as an organic or inorganic solid or liquid excipient suitable for oral, parenteral or external administration. The pharmaceutical preparations may be capsules, tablets, dragees, granules, inhalant, suppositories, solution, lotion, suspension, emulsion, ointment, gel, or the like. If desired, there may be included in these preparations, auxiliary substances, stabilizing agents, wetting or emulsifying agents, buffers and other commonly used additives.

While the dosage of therapeutically effective amount of the compound (I) will vary depending upon the age and condition of each individual patient, an average single dose of about 0.01 mg, 0.1 mg, 1 mg, 10 mg, 50 mg, 100 mg, 250 mg, 500 mg and 1000 mg of the compound (I) may be effective for treating the above-mentioned diseases. In general, amounts between 0.01 mg/body and about 1,000 mg/body may be administered per day.

And further, it was also confirmed that analgesic agent is acceptable and satisfactory to patients if its selectivity of inhibiting activity against COX-I, i.e., cyclooxygenase-II vs. cyclooxygenase-I IC₅₀ values ratio (IC₅₀ against COX-II/IC₅₀ against COX-I) is higher than 30 in a whole blood assay, due to a lack of undesired side effects, such as, gastrointestinal disorders, bleeding, renal toxicity, cardiovascular affection, etc . Until now, no one could know what kind of selectivity should be achieved for producing a clinically acceptable and satisfactory “selective COX-I inhibitor” and no one could produce such kind of “selective COX-I inhibitor”.

Accordingly, one object of this invention is to provide an analgesic agent comprising a selective cyclooxygenase-I inhibitor, a cyclooxygenase-II vs. cyclooxygenase-I IC₅₀ values ratio of which is higher than 30 in a whole blood assay. More preferable selectivity thereof is higher than 50, and most preferable one is higher than 100.

The selectivity of cyclooxygenase-I inhibitors can be determined by analyzing their IC₅₀ values against cyclooxygenase-II and cyclooxygenase-I in a whole blood assay and by calculating IC₅₀ values ratio thereof.

In the present invention, the “whole blood assay” means an assay method by using whole blood, particularly human whole blood The inhibiting activity of test compounds against COX-I can be confirmed by assaying the inhibition of TXB₂ production in a human whole blood. And the inhibiting activity of test compounds against COX-II can be confirmed by assaying the inhibition of PGE₂ in a human whole blood.

Details thereof are shown by “[B] Inhibiting activity against COX-I and COX-II” in the present application. And, the selectivity of test compounds against COX-I and COX-II can be confirmed thereby.

In addition to the above IC₅₀ values ratio, it is preferable that the cyclooxygenase-II IC₅₀ value of “selective cyclooxygenase-I inhibitor” is higher than 0.2 μM in whole blood assay, more preferably higher than 0.5 μM, and most preferably higher than 1.0 μM, in order to remove the effect of COX-II inhibiting activity.

The present invention also provides a method for selecting a cyclooxygenase-I inhibitor that lacks gastrointestinal disorders, by assessing whether cyclooxygenase-II vs. cyclooxygenase-I IC₅₀ values ratio is higher than 30, more preferably higher than 50, and most preferably higher than 100, in whole blood assay.

In order to prove the above invention in more details, the following pharmacological data are shown

[1] Selective Inhibiting Activity Against COX-I in Whole Blood Assay

IC₅₀ values of various test compounds were obtained according to a similar manner to the test method shown in “[B] Inhibiting activity against COX-I and COX-II” described in the above. And their selectivity against COX-I was assessed by calculating cyclooxygenase-II vs. cyclooxygenase-I IC₅₀ values ratio. The results are shown in Table 1.

[2] Analgesic Activity:

Effect on Adjuvant Arthritis in Rats:

Test Method (the same as [A]):

Arthritis was induced by injection of 0. 5 mg of Mycobacterium tuberculosis (Difco Laboratories, Detroit, Mich.) in 50 μl of liquid paraffin into the right hind footpad of Lewis rats aged 7 weeks. Analgesic activity of a single dose of agents in arthritic rats was studied. Arthritic rats were randomized and grouped (n=10) for drug treatment based on pain threshold of left hind paws and body weight on day 22. Drugs (Test compounds) were administered and the pain threshold was measured 2 hr after drug administration. The intensity of hyperalgesia was assessed by the method of Randall-Selitto. The mechanical pain threshold of the left hind paw (uninjected hind paw) was determined by compressing the ankle joint with a balance pressure apparatus (Ugo Basile Co. Ltd, Varese, Italy) The threshold pressure of rats squeaking or struggling was expressed in grams. The threshold pressure of rats treated with drugs was compared with that of non-treated rats. The ratio was shown in Table 1. A dose showing the ratio of 1.5 is considered to be the effective dose.

[3] Stretching Test:

Male ddY mice were used after a 24 h fast. Drugs were orally administered to groups of 10 mice. Mice were injected intraperitoneally (i.p.) with 0.2 ml/10 g of 0.690 acetic acid 1 h after the drug administration and then placed singly in a plastic animal cage. Stretching responses, defined as constriction of the abdomen with stretching of the hind limbs, were counted for 10 min from 3 min after the i.p. injection of acetic acid. The results are shown in Table 1.

[4] Gastric Ulcerogenic Activity in Rats:

Male Sprague-Dawley rats were used after a 24 h fast. Drugs were orally administered to groups of 10 rats 5 h before autopsy. The stomachs were macroscopically inspected and scored as follows: 0, no evidence of gastric lesions; 1, spotty submucosal hemorrhage or appearance of erosion; 3, widespread adherence of blood and large areas of submucosal hemorrhage or one to four small ulcers; 4, more than four small ulcers or one large ulcer; 5, numerous large ulcers. The results are shown in Table 1.

TABLE 1

| | | | | Analgesic | | Gastric |
|---|---|---|---|---|---|---|
| | | | | activity in | Inhibiting | ulcerogenic |
| | | | | adjuvant | activity in | activity |
| Test | | | Select- | arthritis | stretching | (Non-toxic |
| compound | COX-I | COX-II | ivity | −3.2 mg/Kg- | test | dose) |
| A | 0.011 | 8.5 | 770 | 1.62 | 68% | >100 mg/Kg |
| | | | | | (32 mg/Kg) | |
| B | 0.015 | 3.8 | 253 | 1.54 | 68% | >100 mg/Kg |
| | | | | | (32 mg/Kg) | |
| C | 0.017 | 1.9 | 112 | 1.57 | 48% | >100 mg/Kg |
| | | | | | (10 mg/Kg) | |
| D | 0.012 | 0.65 | 54 | 1.59 | 58% | >100 mg/Kg |
| | | | | | (10 mg/Kg) | |
| References | | | | | | |
| E | 0.0024 | 0.10 | 42 | — | — | <100 |
| F | 0.011 | 0.15 | 14 | — | — | <100 |
| G | 0.054 | 0.21 | 3.9 | — | — | 3.2 |
| H | 0.42 | 0.63 | 1.5 | — | — | 10 |
| I | 0.18 | 0.19 | 1.1 | — | — | 1 |
| J | 0.15 | 0.028 | 0.19 | — | — | 3.2 |

(“C”: Compound produced by Example 4 in the present invention, “F”:SC-560, “G”: Ketoprofen, “H”: Mofezolac, “I”: Indomethacin, “J”: Diclofenac)

From the above experimental data, SC-560 and Ketoprofen, still show insufficient selectivity of inhibiting activity against COX-I and thereby gastrointestinal disorders, though they are announced as “selective COX-I inhibitor” in general. And it was confirmed that the selectivity of inhibiting activity against COX-I, i.e., IC₅₀ values ratio, should be more than 30, and that cyclooxygenase-II IC₅₀ value should be higher than 0.2 μM in whole blood assay.

In other word, the selective cyclooxygenase-I inhibitor, that (1) has a cyclooxygenase-II vs. cyclooxygenase-I IC₅₀ values ratio higher than 30 in whole blood assay, and (2) has the cyclooxygenase-II IC₅₀ value higher than 0.2 M in whole blood assay, shows excellent analgesic activity without causing undesired side effects, such as gastrointestinal disorders.

In the present invention, the more preferable analgesic agent is the one comprising the selective cyclooxygenase-I inhibitor, that (1) has a cyclooxygenase-II vs. cyclooxygenase-II IC₅₀ values ratio higher than 50, more preferably 100, in whole blood assay, and that (2) has the cyclooxygenase-II IC₅₀ value higher than 0.5 μM, more preferably 1.0 μM, in whole blood assay.

Accordingly, the analgesic agent of this invention are useful for treating or preventing acute or chronic pains caused by or associated with acute or chronic inflammations in human beings or animals by using administered systemically or topically.

Particularly, the following diseases are exemplified: pains caused by or associated with rheumatoid arthritis, osteoarthritis, lumbar rheumatism, rheumatoid spondylitis, gouty arthritis, juvenile arthritis, etc; lumbago; cervico-omo-brachial syndrome; scapulohumeral periarthritis; pain and tumescence after operation or injury; etc.

For therapeutic purpose, the analgesic agent of the present invention can be used in a form of pharmaceutical preparation suitable for oral, parenteral or external administration. The pharmaceutical preparations may be capsules, tablets, dragees, granules, inhalant, suppositories, solution, lotion, suspension, emulsion, ointment, gel, or the like.

The dosage of therapeutically effective amount of the analgesic agent will vary depending upon the age and condition of each individual patient.

And further, the present application is concerning the followings.

An article of manufacture, comprising packaging material and the compound (I) identified in claim 1 contained within said packaging material, wherein said the compound (I) is therapeutically effective for preventing or treating inflammatory conditions, various pains, collagen diseases, autoimmune diseases, various immunity diseases, analgesic, thrombosis, cancer or neurodegerative diseases, and wherein said packaging material comprises a label or a written material which indicates that said compound (I) can or should be used for preventing or treating inflammatory conditions, various pains, collagen diseases, autoimmune diseases, various immunity diseases, analgesic, thrombosis, cancer or neurodegerative diseases.

A commercial package comprising the pharmaceutical composition containing the compound (I) identified in claim 1 and a written matter associated therewith, wherein the written matter states that the compound (I) can or should be used for preventing or treating inflammatory conditions, various pains, collagen diseases, autoimmune diseases, various immunity diseases, analgesic, thrombosis, cancer or neurodegerative diseases.

The patents, patent applications and publications cited herein are incorporated by reference.

The following Examples are given for the purpose of illustrating the present invention in detail

### EXAMPLE 1

(1) A mixture of desoxyanisoin (8 g, 31.2 mol) and N,N-dimethylformamide dimethylacetal (9.3 g, 78 mmol) in dimethyl formamide (40 ml) was stirred for 2 hours at 90° C.

The reaction mixture was evaporated under reduced pressure to afford crude I-(N,N-dimethylamino)-2-(4-methoxyphenyl)-3-(4-methoxyphenyl)prop-1-en-3-one (10.72 g) as a yellow solid. The crude solid was used for the next step without further purification.

(2) A mixture of 1-(N,N-dimethylamino)-2-(4-methoxyphenyl)-3-(4-methoxyphenyl)prop-1-en-3-one (10.64 g, 31 mmol) and 2-cyanoacetamide (2.92 g, 34.7 mmol) in N,N-dimethylformamide (80 ml) and methyl alcohol (3 ml) was added to a slurry of NaH (2.73 g, 68.2 mmol: 60% in mineral oil) in N,N-dimethylformamide (40 ml) with cooling by an ice bath. (5 to 18° C.). The reaction mixture was stirred for 12 hr at 80° C. and cooled to room temperature. The resulting mixture was poured into 1 M KH₂PO₄ (400 ml), and filtered, washed with water (100 ml) and dried in vacuo (60° C.) to afford 1,2-dihydro-5-(4-methoxyphenyl)-6-(4-methoxyphenyl)-2-oxo-py ridine-3 carbonitrile (11.83 g) as crystal.

¹H NMR (CDCl₃, δ): 3.80 (3H, s), 3.83 (3H, s), 6.81 (2H, d, J=8.8 Hz), 6.87 (2H, d, J=8.8 Hz), 6.98 (2H, d, J=8.8 Hz), 7.27 (2H, d, J=8.8 Hz), 7.92 (1H, s).

IR (KBr): 2220, 1649, 1606, 1554, 1510, 1464, 1298, 1257, 1180, 1028 cm⁻¹

Mass (ESI): (M+H)⁺ 333.1, (M+Na)⁺ 355.2

(3) A mixture of 1,2-dihydro-5-(4-methoxyphenyl)-6-(4-methoxyphenyl)-2-oxo-py ridine-3-carbonitrile (2 g, 4.85 mmol), phosphorus oxychloride (2.26 ml, 24.3 mmol) and NEt₃ (0.676 ml, 4.85 mmol) was refluxed for 2 hr. The resulting mixture was cooled to room temperature, concentrated under reduced pressure. The residue was dissolved in dichloromethane (10 ml) and 1N hydrochloric acid (10 ml) (exothermic). The organic layer was separated and the aqueous layer was further extracted with dichloromethane (10 ml). The combined extracts were dried over MgSO₄ and concentrated under reduced pressure to afford deep brown solid (1.78 g). The brown solid was purified with column chromatography (Silica gel/Toluene) and triturated with ethyl acetate (3 ml) and concentrated to afford 2-chloro-5-(4-methoxyphenyl)-6-(4-methoxyphenyl) pyridine-3-carbonitrile (1.39 g) as crystal.

¹H NMR (CDCl₃, δ): 3.0 (3H, s), 3.83 (3H, s), 6.79 (2H, d, J=8.9 Hz), 6.87 (2H, d, J=8.8 Hz), 7.10 (2H, d, J=8.8 Hz), 7.37 (2H, d, J=8.9 Hz), 7.90 (1H, s).

IR (KBr): 2223, 1604, 1572, 1512, 1406, 1294, 1252, 1174, 1024 cm⁻¹

Mass (APCI): (M+H)⁺ 351.20

### EXAMPLE 2

2-Chloro-5-(4-methoxyphenyl)-6-(4-methoxyphenyl)-pyridine-3-carbonitrile (5.87 g, 16.7 mmol) was dissolved in dimethyl sulfoxide (64.6 ml) at 60° C. and then cooled to 28° C. by water bath. K₂CO₃ (6.94 g, 50.2 mmol) was added at small portion to the above solution under water bath cooling, successively 30% H₂O₂ (5.87 ml) was added to the reaction mixture (exothermic, 28 to 36° C.). The resulting mixture was stirred for 1 hr under the same condition. The mixture was slowly poured into 1N hydrochloric acid (88.05 ml, 15 v) under ice bath (exothermic, 10 to 25° C.) to afford precipitates. The obtained precipitates were collected, washed with water (59 ml, 5 v) fifth times and dried in vacuo to afford 2-chloro-5-(4-methoxyphenyl)-6-(4-methoxyphenyl)-pyridine-3-carboxamide (5.78 g).

¹H NMR (CDCl₃, δ): 3.80 (3H, s), 3.82 (3H, s), 6.79 (2H, d, J=8.9 Hz), 6.84 (2H, d, J=8.8 Hz), 7.13 (2H, d, J=8.8 Hz), 7.38 (2H, d, J=8.9 Hz), 8.26 (1H, s).

IR (KBr): 1673, 1603, 1579, 1512, 1392, 1292, 1252, 1176, 1024 cm⁻¹

Mass (APCI): (M+H)⁺ 369.20

### EXAMPLE 3

To a mixture 2-chloro-5-(4-methoxyphenyl)-6-(4-methoxyphenyl)-pyridine-3-carboxamide (4.02 g, 10.9 mmol) and NEt₃ (15.2 ml, 109 mmol) in ethyl alcohol (20 ml) and THF (20 ml) was added Pd/C (800 mg). This mixture was hydrogenated for 2 hr at 55° C. and filtrated , washed with THF and ethyl alcohol and concentrated to afford yellow solid. This crust was dissolved in dichloromethane (40 ml, 10 v) and water (40 ml, 10 v) at 50° C. The organic layer was separated, the aqueous layer was further extracted with dichloromethane (20 ml), dried over MgSO₄ and concentrated. The residue was triturated with ethyl acetate (16 ml, 4 v) under reflux for 30 min, cooling to room temperature. The resulting powder was collected, washed with ethyl acetate (8 ml, 2 v) twice and dried in vacuo to afford 5-(4-methoxyphenyl)-6-(4-methoxyphenyl) pyridine-3-carboxamide (2.94 g) as a powder.

¹H NMR (CDCl₃, δ): 3.80 (3H, s), 3.82 (3H, s), 6.80 (2H, d, J=8.8 Hz), 6.84 (2H, d, J=8.8 Hz), 7.14 (2H, d, J=8.8 Hz), 7.36 (2H, d, J=8.8 Hz), 8.12 (1H, d, J=2.2 Hz), 8.99 (1H, d, J=2.2 Hz).

IR (KBr): 1682, 1510, 1383, 1292, 1252, 1176, 1028 cm⁻¹

Mass (APCI):M+H)⁺ 335.20

### EXAMPLE 4

A mixture of 5-(4-methoxyphenyl)-6-(4-methoxyphenyl) pyridine-3-carboxamide (2.89 g, 8.64 mmol) and phosphorus oxychloride (14.2 g, 92.9 mmol) was stirred for 1 hr under reflux The reaction mixture was cooled room temperature, concentrated, codistilled with toluene and the residue was dissolved in ethyl acetate (15 ml), washed with water (10 ml) three times, dried over MgSO₄ and concentrated in vacuo to afford yellow solid. This crust was purified by column chromatography (Silica gel/Toluene: EtOAc=10:1). The obtained powder was recrystallized from n-butyl alcohol (15 ml, 6 v), collected by filtration, washed with n-butyl alcohol (10 ml, 4 v) twice and hexane (10 ml, 4 v) twice and dried in vacuo to afford 5-(4-methoxyphenyl)-6-(4-methoxyphenyl)-pyridine-3-carbonitrile (2 g).

¹H NMR (CDCl₃, δ): 3.80 (3H, s), 3.83 (3H, s), 6.80 (2H, d, J=8.9 Hz), 6.86 (2H, d, J=8.8 Hz), 7.11 (2H, d, J=8.8 Hz), 7.35 (2H, d, J=8.9 Hz), 7.90 (1H, d, J=2.1 Hz), 8.86 (1H, d, J=2.1 Hz).

IR (KBr): 2223, 1581, 1508, 1423, 1292, 1248, 1173, 1022 cm⁻¹

Mass (APCI): (M+H)⁺ 317.40

mp:107-108° C.

### EXAMPLE 5

(1) A mixture of 1,2-dihydro-5-(4-methoxyphenyl)-6-(4-methoxy phenyl)-2-oxo-pyridine-3-carbonitrile (4 g, 9.71 mmol) and KOH (4.32 g) in ethylene glycol (16 ml, 4 v) and water (6 ml, 1.5 v) was heated at 160° C. After stirring for over night, The reaction mixture was cooled to room temperature, and then poured into 1N hydrochloric acid (140 ml) to afford precipitates. The obtained precipitates ware collected by filtration, washed with water twice and dried in vacuo to afford 1,2-dihydro-5-(4-methoxyphenyl)-6-(4-methoxyphenyl)-2-oxo-pyridine-3-carboxylic acid (3.41 g) as a crystal.

¹H NMR (CDCl₃, δ): 3.81 (3H, s), 3.86 (3H, s), 6.82 (2H, d, J=8.7 Hz), 6.90 (2H, d, J=8.8 Hz), 7.05 (2H, d, J=8.7 Hz), 7.29 (2H, d, J=8.8 Hz), 8.62 (1H, s), 12.71 (1H, brs), 13.61 (1H, brs).

IR (KBr): 1728, 1616, 1552, 1506, 1450, 1257, 1176 cm⁻¹

Mass (ESI): (M+Na)⁺ 374.2

(2) 1,2-Dihydro-5-(4-methoxyphenyl)-6-(4-methoxyphenyl).-2-oxo-pyridine-3-carboxylic acid (0.8 g, 2.28 mmol) was heated at 210° C. in quinoline (5 ml). After stirring for over night, ethyl acetate and 1N-hydrochloric acid were added to the reaction mixture. The precipitates were collected by filtration, washed with 1N-hydrochloric acid and ethyl acetate and dried in vacuo to afford 1,2-dihydro-5-(4-methoxyphenyl)-6-(4-methoxyphenyl)-2-oxo-pyridine (1.36 g). The crude solid was used for the next step without further purification.

¹H NMR (CDCl₃, δ): 3.79 (3H, s), 3.81 (3H, s), 6.62 (1H, d, J=9.3 Hz), 6.78 (2H, d, J=8.6 Hz), 6.83 (2H, d, J=8.6 Hz), 6.99 (2H, d, J=8.6 Hz), 7.18 (2H, d, J=8.6 Hz), 7.56 (1H, d, J=9.3 Hz).

Mass (APCI): (M+H)⁺ 308.27

(3) To a solution of 1,2-dihydro-5-(4-methoxyphenyl)-6-(4-methoxyphenyl)-2-oxo-pyridine (0.9 g, 1.6 mmol) in pyridine was added trifluoromethanesulfonic acid anhydride (0.808 ml, 4.8 mmol) and warmed for 1 hr at 60° C. The reaction mixture was concentrated and then purified by column chromatography (Silica gel/40% dichloromethane/hexane) to afford trifuoromethanesulfonic acid 5-(4-methoxyphenyl)-6-(4-methoxyphenyl)-2-yl ester (374 mg)

¹H NMR (CDCl₃, δ): 3.80 (3H, s), 3.82 (3H, s), 6.78 (2H, d, J=8.9 Hz), 6.86 (2H, d, J=8.8 Hz), 7.00-7.20 (3H, m), 7.33 (2H, d, J=8.9 Hz), 7.80 (1H, d, J=8.2 Hz).

IR (KBr): 1603, 1585, 1514, 1446, 1417, 1250, 1174, 1128 cm¹

Mass (APCI): (M+H)⁺ 439.87

(4) A mixture of trifuoromethanesulfonic acid 5-(4-methoxy phenyl)-6-(4-methoxyphenyl)-2-yl ester (80 mg, 0.182 mmol), KCN (35.6 mg, 0.546 mmol), LiCl (23.2 mg, 0.546 mmol), 18crown6 (14 mg , 0.3 eq) and palladium tetrakis (triphenylphosphine) (42.1 mg, 0.0364 mml) in toluene (5 ml) was heated for 15 hr at 100° C. The reaction mixture was cooled to room temperature, and then extracted with EtOAc at several times. The organic layer was washed with water, dried over MgSO₄ and concentrated. The residue was purified by thin layer chromatography (20% Hexane in EtOAc) to afford 5-(4-methoxyphenyl)-6-(4-methoxyphenyl)-pyridine-2-carbonitr ile (27 mg) as crystal.

¹H NMR (CDCl₃, δ) 3.80 (3H, s), 3.82 (3H, s), 6.81 (2H, d, J=8.8 Hz), 6.85 (2H, d, J=8.8 Hz), 7.12 (2H, d, J=8.8 Hz), 7.33 (2H, d, J=8.8 Hz), 7.63 (1H, d, J=7.9 Hz), 7.76 (1H, d, J=7.9 Hz).

IR (KBr): 2233, 1512, 1246, 1174, 1028 cm⁻¹

Mass (APCI): (M+H)⁺ 317.33

### EXAMPLE 6

A mixture of 2-chloro-5-(4-methoxyphenyl)-6-(4-methoxyphenyl)-pyridine-3-carbonitrile (124 mg, 0.353 mmol) and 28% NaOMe in methyl alcohol (5 ml) in N,N-dimethylformamide was refluxed for 2 hr. The reaction mixture was cooled and concentrated under reduced pressure. The residue was diluted with ethyl acetate, washed with water twice and brine, dried over MgSO₄ and concentrated (0.14 g). The crude product was purified by thin layer chromatography (20% Hexane in EtOAc) to afford 2-methoxy-5-(4-methoxyphenyl)-6-(4-methoxyphenyl)-pyridine-3-carbonitrile (0.06 g) as a powder,

¹H NMR (CDCl_{3, δ):} 3.80 (3H, s), 3.82 (3H, s), 4.13 (3H, s), 6.78 (2H, d, J=8.8 Hz), 6.82 (2H, d, J=8.8 Hz), 7.07 (2H, d, J=8.8 Hz), 7.39 (2H, d, J=8.8 Hz), 7.81 (1H, s).

IR (KBr): 2225, 1591, 1462, 1398, 1250, 1173, 1028 cm⁻¹

Mass (APCI): (M+H)⁺ 347.40

### EXAMPLE 7

(1) To a solution of 5-(4-methoxyphenyl)-6-(4-methoxyphenyl)-pyridine-3-carbonitrile (0.13 g, 0.41 mmol) in toluene (5 ml) under nitrogen atmosphere was added DIBAL (0.82 ml:1M in toluene) at −78° C. and stirred for 2 hr at room temperature. The reaction mixture was quenched by 1N hydrochloric acid, basified by sat. NaHCO₃ aq., extracted with ethyl acetate twice, dried over MgSO₄ and concentrated.

The residue was purified by column chromatography (Silica gel/40% EtOAc/hexane) to afford 5-(4-methoxyphenyl)-6-(4-methoxyphenyl)-pyridine-3-carboaldehyde (62 mg).

¹H NMR (CDCl₃, ) 3.81 (3H, s), 3.83 (3H, s), 6.81 (2H, d, J=8.8 Hz), 6.86 (2H, d, J=8.8 Hz), 7.15 (2H, d, J=8.8 Hz), 7.40 (2H, d, J=8.8 Hz), 8.12 (1H, d, J=2.1 Hz), 9.05 (1H, d, J=2.1 Hz), 10.16 (1H, s).

IR (KBr): 1695, 1583, 1512, 1248, 1174, 1028 cm⁻¹

Mass (APCI): (M+H)⁺ 320.33

(2) To a solution of 5-(4-methoxyphenyl)-6-(4-methoxyphenyl)-pyridine-3-carboaldehyde (57 mg, 0.178 mmol) in dichloromethane (5 ml) was added (diethylamino)sulfur trifluoride (86.3 mg, 0.535 mmol) at 0° C. The reaction mixture was stirred for over night at room temperature. The resulting mixture concentrated and purified by thin layer chromatography (30% Hexane in EtOAc) to afford 3-difluoromethyl-5-(4-methoxyphenyl)-6-(4-methoxyphenyl)-pyridine (26 mg).

¹H NMR (CDCl₃, δ): 3.80 (3H, s), 3.82 (3H, s), 6.40-7.10 (5H, m), 7.13 (2H, d, J=8.8 Hz), 7.34 (2H, d, J=8.8 Hz), 7.80 (1H, s), 8.73 (1H, s).

IR (KBr): 1604, 1512, 1452, 1427, 1365, 1252, 1176, 1088, 1032 cm⁻¹

Mass (APCI): (M+H)⁺ 342.33

### EXAMPLE 8

(1) To a solution of 5-(4-methoxyphenyl)-6-(4-methoxyphenyl)-pyridine-3-carbonitrile (400 mg, 1.26 mmol) in ethyl alcohol (10 ml) and c-hydrochloric acid (600 ul) was added 10% Pd/C (50% wet, 80 mg). The reaction mixture was hydrogenated for 2 hr at 55° C. The resulting mixture was filtered and concentrated. The residue was resolved in ethyl acetate and 1N hydrochloric acid aq. The aqueous layer was separated and the organic layer was further extracted with 1N hydrochloric acid aq. The combined hydrochloric acid layer was basified by 1N NaOH aq. and extracted with dichloromethane three times. The combined organic layer was dried over MgSO₄ and concentrated. The residue was purified by column chromatography (Silica gel/15% methyl alcohol/CH₃Cl) to afford 5-(4-methoxyphenyl)-6-(4-methoxyphenyl)-pyridine-3-methyl-amine (305 mg).

¹H NMR (CDCl₃, δ) 3.79 (3H, s), 3.81 (3H, s), 3.97 (2H, s), 6.78 (2H, d, J=8.9 Hz), 6.82 (2H, d, J=8.9 Hz), 7.12 (2H, d, J=8.9 Hz), 7.30 (2H, d, J=8.9 Hz), 7.65 (1H, d, J=2.2 Hz), 8.57 (1H, d, J=2.2 Hz).

IR (neat): 1608, 1512, 1292, 1242, 1178, 1032 cm⁻¹

Mass (APCI): (M+H)⁺ 321.33

(2) To a mixture of 5-(4-methoxyphenyl)-6-(4-methoxyphenyl)-pyridine-3-methylamine (139 mg, 0.434 mmol) and 35% HCHOaq. (12.6M, 344 ul) in dichloromethane (5 ml) and methyl alcohol (2 ml) was added NaBH(OAc)₃ (552 mg, 2.6 mmol)at room temperature and then stirred for 30 min. The reaction was quenched with water, extracted with dichloromethane twice, dried over MgSO₄ and concentrated. The residue was purified by thin layer chromatography (10% methyl alcohol in dichloromethane) to afford an oil.

This oil was dissolved in dichloromethane (5 ml) was treated with 4N hydrochloric acid in ethyl acetate (1 ml) (suspension) and concentrated. The hydrochloric acid salts were triturated with dichloromethane and iso-propyl ether and concentrated to afford 5-(4-methoxyphenyl)-6-(4-methoxyphenyl)-pyridine-3-N,N-dimethylmethylamine hydrochloride (98 mg

¹H NMR (CDCl₃, δ) 3.00 (6H, s), 3.81 (3H, s), 3.85 (3H, s), 4.60-5.00 (2H, m), 6.70-7.80 (8H, m), 9.37 (1H, s), 9.52 (1H, s).

IR (KBr): 1606, 1510, 1252, 1182, 1024 cm⁻¹

Mass (APCI): (M+H)⁺ (free) 349.27

### EXAMPLE 9

A mixture of 5-(4-methoxyphenyl)-6-(4-methoxyphenyl)-pyridine-3-methylamine (96 mg, 0.3 mmol) and methylisocyanate (25.3 mg, 0.449 mmol) in THF (5 ml) and methyl alcohol (1 ml) was stirred for 1 hr at room temperature. The reaction was concentrated and the residue was purified by thin layer chromatography (10% methyl alcohol in dichloromethane) to afford an oil. This oil was treated with 4N hydrochloric acid in ethyl acetate and concentrated. The residue was triturated with dichloromethane/iso-propyl ether to afford as a pale yellow powder N-5-(4-methoxyphenyl)-6-(4-methoxyphenyl)-3-methylpyridine-N′-methylurea hydrochloride (95 mg).

¹H NMR (CDCl₃, δ): 2.75 (3H, s), 3.82 (6H, s), 4.64 (2H, s), 6.85 (2H, d, J=3.1 Hz), 6.89 (2H, d, J=3.1 Hz), 7.10 (2H, d, J=8.6 Hz), 7.38 (2H, d, J=8.6 Hz), 8.35 (1H, brs), 8.95 (1H, brs).

IR (KBr): 2058, 1652, 1606, 1510, 1460, 1302, 1255, 1180 cm⁻¹

Mass (APCI): (M+H)^{+ (free)} 378.00

### EXAMPLE 10

To a solution of 5-(4-methoxyphenyl)-6-(4-methoxyphenyl)-pyridine-3-carbonitrile (24 mg, 0.076 mmol) in ethyl alcohol (5 ml) and c-hydrochloric acid (40 μl) was added 10% Pd/C (50% wet, 10 mg) The reaction mixture was hydrogenated for 2.5 hr at 55° C. The resulting mixture was filtrated and concentrated to afford 5-(4-methoxyphenyl)-6-(4-methoxyphenyl)-pyridine-3-methylamine hydrochloride (31 mg) as pale yellow powder.

¹H NMR (CDCl₃, δ): 3.68 (3H, s), 3.71 (3H, s), 3.84 (2H, s), 6.40-8.00 (10H, m).

IR (KBr): 1605, 1510, 1257, 1180, 1022 cm⁻¹

Mass (APCI): (M+H)⁺ 321.27

### EXAMPLE 11

(1) A mixture of 3-bromo-5-methyl-2-aminopyridine (4.0 g, 21.4 mmol), 4-methoxybenzeneboronic acid (3.9 g, 25.7 mmol) and palladium tetrakis (triphenylphosphine) (247 mg, 0.214 mmol) in benzene (20 ml)-ethylalcohol (20 ml)-2M Na₂CO₃ (24 ml) was refluxed for 16 hr. The reaction mixture was diluted with ethyl acetate and water and the organic layer was separated. The aqueous layer was further extracted with ethyl acetate. The combined organic layer was dried over MgSO₄ and concentrated. The residue was purified by column chromatography (Silica gel/20-70% ethyl acetate/hexane) to afford 5-methyl-3-(4-methoxyphenyl)-2-aminopyridine (4.71 g)

¹H NMR (CDCl₃, δ): 2.22 (3H, s), 3.85 (3H, s), 4.44 (2H, brs), 6.98 (2H, d, J=8.8 Hz), 7.18 (1H, d, J=2.1 Hz), 7.37 (2H, d, J=8.8 Hz), 7.88 (1H, s, J=2.1 Hz).

IR (KBr): 1627, 1610, 1568, 1508, 1464, 1406, 1284, 1244, 1173, 1026 cm⁻¹

Mass (APCI): (M+H)⁺ 215.27

(2) To a mixture of 5-methyl-3-(4-methoxyphenyl)-2-aminopyridine (1 g, 4.57 mmol) in ethyl alcohol (10 ml)-1.88M H₂SO₄ solution was added n-butyl alcohol (10 ml), NaNO₂ (5.15 g, 74.7 mmol) and then the resulting mixture was stirred for 4 h at 65° C. The mixture was diluted with ethyl acetate and water and the organic layer was separated. The aqueous layer was further extracted with ethyl acetate. The combined organic layer was dried over MgSO₄ and concentrated. The residue was triturated with ethyl acetate and iso-propyl ether, collected by filtration, washed with iso-propyl ether and dried in vacuo to afford 5-methyl-3-(4-methoxyphenyl)-2-hydroxypyridine (0.55 g) as pale orange powder.

¹H NMR (CDCl₃, δ): 2.13 (3H, s), 3.85 (3H, s), 6.96 (2H, d, J=8.8 Hz), 7.12 (1H, d, J=2.4 Hz), 7.41 (1H, d, J=2.4 Hz), 7.68 (2H, d, J=8.8 Hz), 12.64 (1H, brs).

IR (KBr): 1657, 1562, 1510, 1290, 1246, 1173, 1024 cm⁻¹

Mass (APCI): (M+H)⁺ 216.20

(3) Trifuoromethanesulfonic acid 3-methyl 3-(4-methoxyphenyl) pyridine-2-yl ester was prepared from 5-methyl-3-(4-methoxyphenyl)-2-hydroxypyridine by the similar method as that described for Example 5-(3).

¹H NMR (CDCl₃, δ): 2.41 (3H, s), 3.87 (3H, s), 7.00 (2H, d, J=8.8 Hz), 7.40 (2H, d, J=8.8 Hz), 7.66 (1H, d, J=2.0 Hz), 8.11 (1H, d, J=2.0 Hz).

IR (neat): 1610, 1516, 1414, 1252, 1215, 1140, 1038 cm⁻¹

Mass (ESI): (M+H)⁺ 348.1, (M+Na)⁺ 370.1

(4) 2-Methyl-5-(4-methoxyphenyl)-6-(4-methoxyphenyl) pyridine hydrochloride was prepared from trifuoromethanesulfonic acid 3-methyl 3-(4-methoxyphenyl) pyridine-2-yl ester by the similar method as that described for Example 11-(1).

¹H NMR (CDCl₃, δ): 2.56 (3H, s), 3.83 (6H, s), 6.87 (2H, d, J=2.9 Hz), 6.91 (2H, d, J=2.9 Hz), 7.11 (2H, d, J=8.8 Hz), 7.46 (2H, d, J=8.8 Hz), 8.06 (1H, s), 8.73 (1H, s).

IR (KBr): 2089, 1606, 1508, 1460, 1255, 1178, 1024 cm⁻¹

Mass (APCI): (M+H)^{+ (free)} 306.20

### EXAMPLE 12

(1) 2-Benzyloxy-5-chloro-3-(4-methoxyphenyl) pyridine was prepared from 2-benzyloxy-3-bromo-5-chloropyridine by the similar method as that described for Example 11-(1)

¹H NMR (CDCl₃, δ): 3.85 (3H, s), 5.44 (2H, s), 6.95 (2H, d, J=8.9 Hz), 7.20-7.50 (5H, m), 7.53 (2H, d, J=8.9 Hz), 7.60 (1H, d, J=2.6 Hz), 8.05 (1H, d, J=2.6 Hz).

IR (KBr): 1608, 1510, 1435, 1362, 1302, 1244, 1174, 1032 cm⁻¹

Mass (APCI): (M+H)⁺ 326.13

(2) A mixture of 2-benzyloxy-5-chloro-3-(4-methoxyphenyl) pyridine in 6N hydrochloric acid (10.5 ml) and ethyl alcohol/toluene (1/1, 10.5 ml) was refluxed for 2 h. The reaction mixture was cooled to room temperature and diluted with ethyl acetate and water. The organic layer was separated and the aqueous layer was further extracted with dichloromethane. The crystal was collected by filtration (5-chloro-3-(4-methoxyphenyl)-2-hydroxypyridine: 0.32 g), washed with ethyl acetate and dried in vacuo. The combined filtrate was dried over MgSO₄ and concentrated. The residual solid was triturated with iso-propyl ether, collected by filtration, washed with iso-propyl ether and dried in vacuo to afford 5-chloro-3-(4-methoxyphenyl)-2-hydroxypyridine (0.98 g).

¹H NMR (DMSO-d6, δ): 3.78 (3H, s), 6.96 (2H, d, J=8.9 Hz), 7.56 (1H, d, J=2.9 Hz), 7.62 (1H, d, J=2.9 Hz), 7.72 (2H, d, J=8.9 Hz), 11.98 (1H, brs).

IR (KBr): 1651, 1604, 1510, 1468, 1250, 1178, 1022 cm⁻¹

(3) Trifuoromethanesulfonic acid 5-chloro-3-(4-methoxyphenyl) pyridine-2-yl ester was prepared from 5-chloro-3-(4-methoxyphenyl)-2- hydroxypyridine by the similar method as that described for Example 5-(3).

¹H NMR (CDCl₃, δ): 3.87 (3H, s), 7.02 (2H, d, J=8.8 Hz), 7.41 (2H, d, J=8.8 Hz), 7.85 (1H, d, J=2.5 Hz), 8.25 (1H, d, J=2.5 Hz).

IR (neat): 1610, 1516, 1421, 1252, 1217, 1140, 1034 cm⁻¹

Mass (ESI): (M+H)⁺ 368.0, (M+Na)⁺ 390.1

(4) 5-Chloro-2-(4-methoxyphenyl)-3-(4-methoxyphenyl) pyridine was prepared from trifuoromethanesulfonic acid 5-chloro-3-(4-methoxyphenyl) pyridine-2-yl ester (XVII) by the similar method as that described for Example 11-(1).

1H NMR (CDCl₃, δ): 3.79 (3H, s), 3.81 (3H, s), 6.78 (2H, d, J=8.8 Hz), 6.83 (2H, d, J=8.8 Hz), 7.10 (2H, d, J=8.8 Hz), 7.28 (2H, d, J=8.8 Hz), 7.66 (1H, d, J=2.4 Hz), 8.57 (1H, d, J=2.4 Hz).

IR (neat): 1608, 1512, 1460, 1429, 1246, 1178, 1115, 1030 cm⁻¹

Mass (ESI): (M+H)⁺ 326.3, (M+Na)⁺ 348.1

### EXAMPLE 13

(1) To a suspension of 6-amino-nicotinamide (4.45 g, 32.4 mmol) in acetic acid (100 ml) was added dropwise bromine (1.84 ml, 35.7 mmol))at room temperature. The mixture was stirred at 55° C. for 3 hours. The resulting solution was added to 750 ml of 3N aqueous sodium hydroxide. The resulting sediment was filtered, washed with water and was dried over to afford 6-amino-5-bromonicotinamide. This was used for the next step without further purification.

¹H-NMR (DMSO-d6, δ); 6.77 (2H, br), 7.19 (1H, br), 7.77 (1H, br), 8.15 (1H, d, J=2.0 Hz), 8.47 (1H, d, J=2.0 Hz),

MASS (APCI); 216 (M+H)+

(2) To the solution of 6-amino-5-bromonicotinamide (6.67 g, 30.9 mmol), 4-methoxybenzeneboronic acid (5.63 g, 37 mmol) and tetrakis(triphenylphosphin)-palladium (0) (1.78 g, 1.54 mmol)in ethylene glycol dimethylether (60 ml) was added 2M aqueous sodium carbonate (92 ml). And this was stirred for 15 hours at 100° C. After the mixture was cooled to room temperature, this was diluted with 1N aqueous sodium hydroxide. The reaction mixture was extracted with ethyl acetate. The extract was washed with brine, dried over magnesium sulfate and evaporated under reduced pressure. The resulting residue was purified by silica gel column chromatography with a mixture of hexane and ethyl acetate (1:1) as an eluent to afford 6-amino-5-(4-methoxyphenyl)-nicotinamide (5.97 g, white solid).

¹H-NMR (DMSO-d6, δ); 3.80 (3H, s), 6.09 (2H, s), 7.04 (3H, m, J=8.8 Hz), 7.38 (3H, d, J=8.8 Hz), 7.75 (29H, m), 8.47 (1H, d, J=1.9 Hz),

MASS (ESI); 266 (M+Na)+

(3) 6-Amino-5-(4-methoxyphenyl)nicotinamide (700 mg, 2.88 mg) was dissolved into acetone (15 ml) and 1.88 M H₂SO₄ (15 ml). To this solution, 5 M NaNO₂ (5.8 ml) was added dropwise under ice cooling. And the resultant solution was stirred for 5 h. (Bubbled, Brown gas). Another NaNO₂ (567 mg, in 2 ml water) was added at 0° C. and was stirred for 3 h. Sediment was filtrated, washed with water and dried up to give 6-hydroxy-5-(4-methoxyphenyl)-nicotinamide (543 mg). This was used for the next step without further purification.

(4) To the solution of 6-hydroxy-5-(4-methoxyphenyl)-nicotinamide (1.8 g, 7.37 mmol) and triethylamine hydrochloride (7.1 g, 51.6 mmol) in toluene was added phosphorous chloride. After being stirred for 12 hours at 110° C., the reaction mixture was poured into water, extracted with ethyl acetate. The extract was washed with brine, dried over magnesium sulfate and evaporated under reduced pressure. The resulting residue was purified by silica gel column chromatography with a mixture of hexane and ethyl acetate (2:1) as an eluent to afford 6-chloro-5-(4-methoxyphenyl)nicotinonitrile.

¹H-NMR (CDCl₃, δ); 3.88 (3H, s), 7.01 (2H, d, J=9.7 Hz), 7.39 (2H, d, J=9.7 Hz), 7.90 (1H, d, J=2.3 Hz), 8.62 (1H, d, J=2.3 Hz),

IR (cm⁻¹); 1693, 1617, 1515, 1380, 1251, 1186, 1095, 1025, 827

MASS (APCI): 245 (M+H)+

(5) To the solution of 6-chloro-5-(4-methoxyphenyl)-nicotinonitrile (220 mg, 0.899 mmol) in dimethoxyethane (5 ml), 6-methoxy-3-pyridinylboronic acid (344 mg, 2.25 mmol), tetrakis(triphenylphosphin)palladium (31.2 mg, 0.027 mmol) and 2M Na₂CO₃ (1.8 ml, 36 mmol) was added. The reaction mixture was stirred at 80° C. for 12 h.

This was diluted with ethyl acetate, washed with 0.1N hydrochloric acid and brine. After subjecting an extraction with ethyl acetate, a purification by silica gel column chromatography was carried out with hexane/ethyl acetate (5-3/1) as an eluent. And 5-(4-methoxyphenyl)-6-(6-methoxypyridine-3-yl)-nicotinonitri le (colorless crystal needle) was obtained by recrystalization from ethyl alcohol.

IR (KBr, cm⁻¹); 2225, 1602, 1575, 1504, 1438, 1400, 1371, 1309, 1292, 1245, 1174, 1118, 1064, 1014, 939, 827, 786

¹H-NMR (CDCl₃, δ); 3.83 (3H, s), 3.93 (3H, s), 6.64 (1H, d, J=8.7 Hz), 6.88 (2H, d, J=9 Hz), 7.1 (2H, d, J=9 Hz), 7.6 (1H, dd, J=8.725 Hz), 7.92 (1H, d, J=2.1 Hz), 8.22 (1H, d, J=2.5 Hz), 8.88 (1H, d, J=2.1 Hz),

MASS (APCI); 318 (M+H)+

### EXAMPLE 14

A mixture of the compound obtained in a similar manner to that of Example 1-(3) (7.02 g, 20 mmol) and zinc powder (5.26 g) in a mixture of acetic acid (80 mL) and N,N-dimethylformamide (40 mL) was stirred at 55° C. for 8 h.

Then the reaction mixture was stirred at room temperature for 14 h. Zinc salt was removed by filtration. To the filtrate was added toluene (200 mL) and water (100 mL). The layers were separated and the aqueous layer was re-extracted with toluene (100 mL). The combined organic layer was washed with water (100 mL) and 10% brine (100 mL) respectively. The organic layer was evaporated and the residual waxy oil was treated with 50% ethyl alcohol in water. The precipitate was collected and dried under reduced pressure to afford the same compound as Example 4 (5.37 g, 85% yield) as an yellowish solid.

¹H-NMR (CDCl₃, δ); 3.80 (s, 3H), 3.82 (s, 3H), 4.60 (s, 2H), 6.79 (dd, 2H, J=8.9, 2.0), 6.86 (dd, 2H, J=8.7, 2.0), 7.11 (dd, 2H, J=8.8, 2.1) 7.35 (dd, 2H, J=8.8, 2.1), 7.89 (d, 1H, J=2.1), 8.85 (d, 1H, J=2.0);

MS (EI); m/z 317.3 (M+H)+

The chemical structures of the compounds produced by the above Examples are listed in the following Table 2.

TABLE 2

| Example No. | R¹ | R² | R³ | R⁴ |
|---|---|---|---|---|
| 1-(3) | —CN | —Cl | 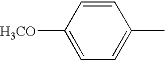 | —OCH₃ |
| 2 | —CONH₂ | —Cl | 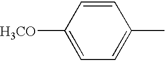 | —OCH₃ |
| 3 | —CONH₂ | —H | 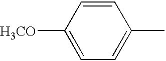 | —OCH₃ |
| 4 | —CN | —H | 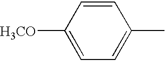 | —OCH₃ |
| 5-(4) | —H | —CN | 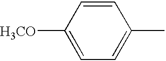 | —OCH₃ |
| 6 | —CN | —OCH₃ | 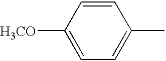 | —OCH₃ |
| 7-(1) | —CHO | —H | 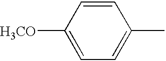 | —OCH₃ |
| 7-(2) | —CHF₂ | —H | 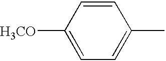 | —OCH₃ |
| 8-(1) | —CH₂NH₂ | —H | 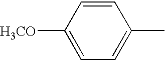 | —OCH₃ |
| 8-(2) | —CH₂N(CH₃)₂ | —H | 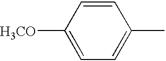 | —OCH₃ |
| 9 | —CH₂NH-CO-NHCH₃ | —H | 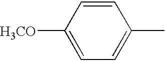 | —OCH₃ |
| 10 | —CH₂NH₂ | —H | 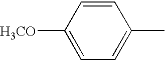 | —OCH₃ |
| 11-(4) | —CH₃ | —H | 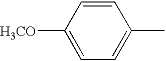 | —OCH₃ |
| 12-(4) | —Cl | —H | 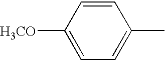 | —OCH₃ |
| 13-(5) | —CN | —H | 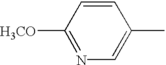 | —OCH₃ |
| 14 | —CN | —H | 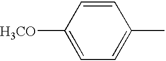 | —OCH₃ |

1. A compound of the formula (I): 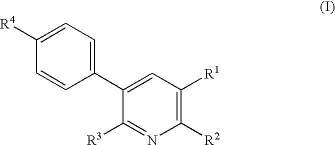
wherein
R¹ is hydrogen, halogen, carbamoyl, cyano, formyl, or lower alkyl optionally substituted with halogen, amino or a protected amino;
R²is hydrogen, halogen, cyano or lower alkoxy;
R³ is phenyl or pyridyl, each of which is substituted with lower alkoxy; and
R⁴ is lower alkoxy;
provided that either R¹ or R² is hydrogen, then the other is other than hydrogen,
or its salts.

2. The compound of claim 1, in which
R¹ is halogen, carbamoyl, cyano, formyl, or lower alkyl optionally substituted with halogen, amino or a protected amino;
R² is hydrogen;
R³ is phenyl substituted with lower alkoxy, or pyridyl substituted with lower alkoxy; and
R⁴ is lower alkoxy.

3. The compound of claim 1, in which
R¹ is hydrogen;
R² is halogen, cyano or lower alkoxy;
R³ is phenyl substituted with lower alkoxy; and
R⁴ is lower alkoxy.

4. The compound of claim 1, in which
R¹ is cyano, or lower alkyl optionally substituted with halogen, amino or a protected amino;
R² is halogen, cyano or lower alkoxy;
R³ is phenyl substituted with lower alkoxy; and
R⁴ is lower alkoxy.

5. A pharmaceutical composition comprising the compound (I) or its salts of claim 1, as an active ingredient, in association with a pharmaceutically non-toxic carrier or excipient.

6. The composition of claim 5, which is for treating and/or preventing inflammatory conditions, various pains, collagen diseases, autoimmune diseases, various immunity diseases, analgesic, thrombosis, cancer or neurodegerative diseases

7. A compound of claim 1 for use as a medicament.

8. A method for treatment and/or prevention of inflammatory conditions, various pains, collagen diseases, autoimmune diseases, various immunity diseases, analgesic, thrombosis, cancer or neurodegerative diseases which comprises administering an effective amount of the compound (I) or its salts of claim 1 to human beings or animals.

9. A use of the compound of claim 1 for the manufacture of a medicament for treatment and/or prevention of inflammatory conditions, various pains, collagen diseases, autoimmune diseases, various immunity diseases, analgesic, thrombosis, cancer or neurodegerative diseases in human beings or animals.

10. An analgesic agent comprising a selective cyclooxygenase-I inhibitor, a cyclooxygenase-II vs. cyclooxygenase-I IC₅₀ values ratio of which is higher than 30 in a whole blood assay and cyclooxygenase-II IC₅₀ value thereof is higher than 0.2 μM in a whole blood assay.

11. The analgesic agent of claim 10, which is usable for treating and/or preventing pains caused by or associated with acute or chronic inflammations without causing gastrointestinal disorders.

12. The analgesic agent of claim 10, which is usable for treating or preventing pains caused by or associated with rheumatoid arthritis, osteoarthritis, lumbar rheumatism, rheumatoid spondylitis, gouty arthritis, or juvenile arthritis; lumbago; cervico-omo-brachial syndrome; scapulohumeral periarthritis; pain and tumescence after operation or injury without causing gastrointestinal disorders.

13. The analgesic agent of claim 10, in which the IC₅₀ values ratio is higher than 50.

14. The analgesic agent of claim 10, in which the IC₅₀ values ratio is higher than 100.

15. The analgesic agent of claim 10, in which the cyclooxygenase-II IC₅₀ value of the selective cyclooxygenase-I inhibitor is higher than 0.5 μM.

16. A method for treating and/or preventing inflammatory conditions, various pains, collagen diseases, autoimmune diseases, various immunity diseases, analgesic, thrombosis, cancer or neurodegerative diseases, which comprises administering an effective amount of a selective cyclooxygenase-I inhibitor,
which is characterized by a cyclooxygenase-II vs. cyclooxygenase-I IC₅₀ values ratio of which is higher than 30 in a whole blood assay, and cyclooxygenase-II IC₅₀ value thereof is higher than 0.2 μM in a whole blood assay, to human beings or animals.

17. A use of a selective cyclooxygenase-I inhibitor,
which is characterized by a cyclooxygenase-II vs. cyclooxygenase-I IC₅₀ values ratio of which is higher than 30 in a whole blood assay and cyclooxygenase-II IC₅₀ value thereof is higher than 0.2 μM in a whole blood assay,
for the manufacture of a medicament for treatment and/or prevention of inflammatory conditions, various pains, collagen diseases, autoimmune diseases, various immunity diseases, analgesic, thrombosis, cancer or neurodegerative diseases.

18. A method for selecting a selective cyclooxygenase-I inhibitor, which lacks gastrointestinal disorders, by assessing whether cyclooxygenase-II vs. cyclooxygenase-I IC₅₀ values ratio is higher than 30 in a whole blood assay and whether the cyclooxygenase-II IC₅₀ value thereof is higher than 0.2 μM in whole blood assay.
